(19) United States
(12) Patent Application Publication
(10) Pub. No.: US 2007/0093717 A1
(43) Pub. Date: Apr. 26, 2007

(54) Wearable glucometer configurations

(75) Inventors: Ron Nagar, Tel-Aviv (IL); Gabriel Bitton, Jerusalem (IL); Benny Pesach, Rosh-Ha'ayin (IL); Moshe Ben-David, Ael Aviv (IL)

Correspondence Address: WOLF, BLOCK, SCHORR & SOLIS-COHEN LLP, 250 PARK AVENUE, NEW YORK, NY, 10177, US

(73) Assignee: Glucon Inc., Boulder, CO (US)

(21) Appl. No.: 11/255,064

(22) Filed: Oct. 20, 2005

(51) Int. Cl. A61B 8/00; A61B 5/00

(52) U.S. Cl. 600/438; 600/310

(57) ABSTRACT

Apparatus for assaying an analyte in a patient's body comprising: at least one sensor unit mountable to a region of the patient's skin comprising at least one light source that illuminates a tissue region below the skin with light that generates photoacoustic waves therein and at least one acoustic transducer that generates signals responsive to the photoacoustic waves; and a control unit configured to be mounted to a different part of the body and comprising a controller for controlling the at least one light source and at least one acoustic transducer and receiving the signals generated by the at least one acoustic transducer.

### FIELD OF THE INVENTION

The invention relates to wearable apparatus that can be coupled to a body and continuously assay a substance in the body for an extended period of time and in particular wearable apparatus for continuously monitoring glucose levels in a body.

### BACKGROUND OF THE INVENTION

Methods and apparatus for determining blood glucose levels for use in the home, for example by a diabetic who must monitor blood glucose levels frequently, are available. These methods and associated devices are generally invasive and usually involve taking blood samples by finger pricking. Often a diabetic must determine blood glucose levels many times daily and finger pricking is perceived as inconvenient and unpleasant. To avoid finger pricking, diabetics tend to monitor their glucose levels less frequently than is advisable.

Non-invasive in-vivo methods and apparatus for monitoring blood glucose are known. PCT Publication WO 98/38904, the disclosure of which is incorporated herein by reference, describes a “non-invasive, in-vivo glucometer” that uses a photoacoustic effect to measure a person's blood glucose. U.S. Pat. No. 6,846,288, the disclosure of which is incorporated herein by reference, describes locating a blood vessel in the body and determining glucose concentration in a bolus of blood in the blood vessel. The glucose concentration in the blood bolus is determined by illuminating the bolus with light that is absorbed and/or scattered by glucose to generate photoacoustic waves in the bolus. Intensity of the photoacoustic waves, which is a function of glucose concentration, is sensed and used to assay glucose in the bolus.

Wearable devices for assaying glucose are known, are generally based on near-infrared (NIR) spectroscopic methods and usually comprise a light source and optical detector that are attached to a patient's finger, wrist or other part of the body. Wearable NIR devices for assaying glucose are described in U.S. Pat. No. 6,241,663 to Wu, et al. and U.S. Pat. No. 5,551,422, to Simonsen et al., the disclosures of which are incorporated herein by reference.

An apparatus for determining glucose levels is hereinafter referred to as a “glucometer”.

### SUMMARY OF THE INVENTION

An aspect of some embodiments of the present invention relates to providing a wearable glucometer that may be mounted to a patient's skin in alignment with a blood vessel in the patient's body and thereafter operates to repeatedly assay glucose in blood in the blood vessel without requiring substantial user intervention.

It is generally advantageous to determine glucose levels for a patient from blood glucose levels. Prior art wearable glucometers do not in general distinguish between glucose levels in blood and glucose levels in interstitial fluid and cannot therefore assure that glucose assays they provide are blood glucose levels. Unlike prior art wearable glucometers, a glucometer in accordance with an embodiment of the invention provides measurements of glucose levels that are substantially independent of glucose levels in interstitial fluid.

An aspect of some embodiments of the present invention relates to providing a glucometer, which once aligned with a blood vessel will continue to operate properly, providing glucose assays for blood in the blood vessel, in the event that it becomes misaligned by displacements typically encountered during assay operation.

A glucometer in accordance with an embodiment of the present invention comprises an array of acoustic transducers, a light provider, and a controller. The controller controls the light source and the array of transducers to assay glucose in blood in the patient's blood vessel using a photoacoustic effect. To perform the assay, the controller controls the light provider to illuminate a tissue volume defined by a field of view of the glucometer located below the skin to which the glucometer is attached with light that is absorbed and/or scattered by glucose and stimulates photoacoustic waves in the tissue volume. The field of view of the glucometer is defined as a size and location of a volume of tissue below a region of skin to which the glucometer is attached for which the glucometer stimulates photoacoustic waves that are detectable by its transducer array and practically useable to assay glucose in blood in a blood vessel located in the tissue volume. When properly aligned with the blood vessel, a region of the blood vessel is located substantially at the center of the glucometer's field of view. The transducer array generates signals responsive to acoustic energy that is incident on the array from the photoacoustic waves stimulated in the tissue volume.

The controller receives and processes the signals provided by the transducer array to determine which of the signals corresponds to photoacoustic waves originating in the blood vessel and uses those signals in accordance with methods known in the art to assay glucose in blood in the blood vessel. Examples of photoacoustic assay methods useable in the practice of the invention are described in U.S. Pat. No. 6,846,288 referenced above and in PCT Publication WO 2004/086965 the disclosure of which are incorporated herein by reference.

In time, during extended assay operation, a glucometer initially properly aligned with a blood vessel so that a region of the blood vessel is located at the center of the glucometer's field of view, may become misaligned because, for example, of drift in the glucometer position on the skin or because of motion of the skin relative to the blood vessel.

In an embodiment of the invention, the transducer array and light provider are configured so that the field of view of the glucometer is sufficiently large in at least one dimension so that for misalignments typically encountered during assay operation, the blood vessel remains substantially within the glucometer field of view. As a result, assay operation can continue satisfactorily uninterrupted. PCT Publication WO 2005/067786, the disclosure of which is incorporated herein by reference, describes a glucometer having a large field of view.

In an embodiment of the invention, the glucometer is self-aligning and comprises at least one motor and/or actuator coupled to a component of the glucometer, which the motor and/or actuator moves to realign the glucometer if the glucometer determines that it has become misaligned.

In an embodiment of the invention, the glucometer determines that it is misaligned with the blood vessel responsive to a change in assay signals that it receives that cannot be explained by normal changes in blood glucose. For example, the glucometer might determine that amplitude or waveform changes in the signals or a relatively abrupt change in blood glucose level is a result of the glucometer becoming misaligned with the blood vessel and not a result of a change in blood glucose. For a glucometer, in accordance with an embodiment of the invention that can image features below the skin on which it is mounted, such as by using ultrasound or the photoacoustic effect, the glucometer periodically images the features. From the images, the controller determines if the glucometer is or is not aligned with the blood vessel. PCT Publication WO 2004/107971, the disclosure of which is incorporated herein by reference, describes a self-aligning glucometer.

In some embodiments of the invention, to align the glucometer with a blood vessel the controller controls the array of transducers to acoustically image a tissue region in the patient's body beneath the skin. In some embodiments of the invention, to align the glucometer, the controller controls the light provider to illuminate the field of view of the glucometer with light that stimulates photoacoustic waves in the glucometer field of view. The controller processes signals generated by the transducer array responsive to the photoacoustic waves to generate a “photoacoustic image” of features below the skin.

The acoustic and/or photoacoustic image provided by the controller is used to align the glucometer with the blood vessel. Optionally, the controller generates a signal responsive to the acoustic and/or photoacoustic image to aid a user of the glucometer to align the glucometer with the blood vessel. Optionally, the glucometer comprises a display screen and the controller displays the acoustic and/or photoacoustic image, or icons responsive to the images, to facilitate aligning the glucometer with the blood vessel.

According to an aspect of some embodiments of the invention, components of a glucometer, hereinafter referred to as a “split-unit” glucometer, are housed in a plurality of housings. Component units of a split-unit glucometer communicate with each other over wire and/or wireless electrical and/or electromagnetic wave channels. Optionally, component units communicate via at least one acoustic channel such as for example an ultrasound acoustic channel.

Optionally, a split-unit glucometer comprises a sensor unit housed in its own sensor housing, and a control unit housed in its own control housing. Optionally, the sensor unit comprises, housed in its dedicated sensor housing, a light source that provides light for generating photoacoustic waves in tissue below a patient's skin and acoustic transducers for detecting photoacoustic waves generated by the light provided by the light source. The control unit comprises, housed in its dedicated housing, a controller for controlling the light source and acoustic transducers and transmitting and receiving signals to and from the transducers and the light source. The control housing also optionally comprises a power supply for the light source and transducers, a display screen and control buttons for interfacing with the controller.

Splitting a glucometer into at least sensor and control units provides for greater flexibility in configuring the glucometer so that it can be mounted conveniently to a patient's body. Communication and transmission of power between the control and sensor units is optionally over wire and/or wireless electrical and/or electromagnetic wave channels. Optionally, communication between the control and sensor units is via at least one acoustic channel, such as for example an ultrasound acoustic channel.

According to an aspect of some embodiments of the invention, a glucometer or at least one unit of a split-unit glucometer is attached to a patient's skin using a sticker. A new sticker is optionally used to attach the glucometer or unit of the split-unit glucometer each time the glucometer or its unit is to be mounted anew to the patient's skin.

According to an aspect of some embodiments of the invention, the sticker is provided with at least one device for presenting an identifying ID code.

Optionally, the glucometer's controller determines whether a new sticker used to attach a glucometer or glucometer unit to a patient's skin presents a proper ID code. If the sticker does not provide a proper ID code, the controller prevents operation of the glucometer. Optionally, the sticker comprises circuitry such as a transponder, optionally a radio frequency identification (RFID) transponder, for presenting an ID code. Optionally, the sticker is provided with a bar code for presenting an ID code.

In some embodiments of the invention, the sticker is provided with at least one substance, such as for example a dye, toner or ink, having a known absorption and/or scattering cross section for light and/or spatial configuration on or in the sticker, which encodes an ID. The absorption cross-section and/or spatial configuration is read to determine the ID by illuminating the at least one substance with light to stimulate photoacoustic waves in the at least one substance and thereby the sticker that are sensed by transducer, optionally, in the glucometer.

According to an aspect of some embodiments of the invention, the sticker is provided with a light source for stimulating photoacoustic waves in tissue and acoustic transducers for generating signals responsive to the photoacoustic waves. Optionally, the sticker by itself functions as a sensor unit of a split-unit glucometer. Optionally, components of the sticker are formed using at least in part printing methods and devices known in the art.

In some embodiments of the invention, the sticker is formed with a power source for providing energy to circuitry and/or components of the glucometer. Optionally, the power source is formed using a printing technology known in the art such as described in U.S. Pat. No. 6,855,441 the disclosure of which is incorporated herein by reference.

There is therefore provided in accordance with an embodiment of the invention apparatus for assaying an analyte in a patient's body comprising: at least one sensor unit mountable to a region of the patient's skin comprising at least one light source that illuminates a tissue region below the skin with light that generates photoacoustic waves therein and at least one acoustic transducer that generates signals responsive to the photoacoustic waves; and a control unit configured to be mounted to a different part of the body and comprising a controller for controlling the at least one light source and at least one acoustic transducer and receiving the signals generated by the at least one acoustic transducer.

Optionally, each of the control unit and at least one sensor unit comprises a transmitter and receiver for communication over at least one channel. In some embodiments of the invention, the at least one channel comprises a wireless channel. Optionally, the at least one wireless channel comprises an electromagnetic wave channel. Optionally, the electromagnetic wave channel comprises an optical channel. Optionally, the at least one wireless channel comprises an acoustic channel.

In some embodiments of the invention, the at least one channel comprises a wire channel. Optionally, the wire channel comprises an optical wave guide. Optionally, the optical wave guide comprises an optical fiber.

In some embodiments of the invention, the control unit transmits operating energy to the at least one sensor over a channel of the at least one channel. Optionally, the at least one sensor comprises a storage unit that stores energy that it receives from the control unit.

In some embodiments of the invention, the at least one sensor unit comprises a power supply.

In some embodiments of the invention, the at least one sensor unit comprises a removable sticker having an adhesive layer that adheres the at least one sensor unit to skin.

In some embodiments of the invention, the sticker comprises a battery. In some embodiments of the invention, the battery is formed at least in part by printing. Optionally, the sticker comprises the at least one light source. Optionally, the at least one light source is formed on or in the sticker at least in part by printing. Optionally, the sticker comprises the at least acoustic transducer. In some embodiments of the invention, the sticker comprises at least acoustic transducer. Optionally, the at least one transducer is formed on or in the sticker at least in part by printing.

In some embodiments of the invention, the sticker comprises a region having an acoustic coupling gel that promotes acoustic coupling of the at least one transducer to skin.

In some embodiments of the invention, the sticker comprises at least one device for presenting an identifying ID code. Optionally, the at least one device comprises a bar code. Optionally, the at least one device comprises circuitry that transmits the ID code when interrogated. Optionally, the circuitry comprises a transponder. Optionally, the transponder comprises a radio frequency ID transponder.

In some embodiments of the invention, the at least one device comprises at least one substance having a known absorption and/or scattering cross section that encodes the ID. In some embodiments of the invention, the at least one device comprises at least one substance characterized by known absorption cross section that is formed on or in the sticker in a spatial configuration that encodes the ID.

In some embodiments of the invention, the controller comprises apparatus to determine whether the at least one device for presenting an identifying ID code presents an acceptable ID code and if it does not, the controller prevents operation of the glucometer.

In some embodiments of the invention, the control unit is mountable to the wrist. In some embodiments of the invention, the sensor unit is configured to be mounted over the femoral artery.

In some embodiments of the invention, the analyte is glucose.

There is further provided in accordance with an embodiment of the invention a method for assaying an analyte in a patient's body comprising: mounting at least one sensor unit to a region of the patient's skin comprising at least one light source that illuminates a tissue region below the skin with light that generates photoacoustic waves therein and at least one acoustic transducer that generates signals responsive to the photoacoustic waves; and mounting to a region of body different from where the sensor unit is mounted, a control unit comprising a controller for controlling the at least one light source and at least one acoustic transducer and processing the signals generated by the at least one acoustic transducer to assay the analyte.

Optionally, mounting the at least one sensor unit comprises mounting the unit over a femoral artery. Optionally, mounting the control unit comprises mounting the unit on the wrist.

### BRIEF DESCRIPTION OF FIGURES

Non-limiting examples of embodiments of the present invention are described below with reference to figures attached hereto, which are listed following this paragraph. In the figures, identical structures, elements or parts that appear in more than one figure are generally labeled with a same numeral in all the figures in which they appear. Dimensions of components and features shown in the figures are chosen for convenience and clarity of presentation and are not necessarily shown to scale.

FIGS. 1A and 1B schematically show a perspective view and cross-section view respectively of a glucometer, in accordance with an embodiment of the present invention;

FIG. 2A schematically show a sticker suitable for attaching a glucometer to a person's skin, in accordance with an embodiment of the invention;

FIG. 2B schematically shows a sticker similar to the sticker shown in FIG. 2A and comprising an RFID transponder in accordance with an embodiment of the invention;

FIG. 2C schematically shows a sticker similar to the sticker shown in FIG. 2A and comprising a bar code, in accordance with an embodiment of the invention;

FIG. 3 schematically shows a glucometer adapted to be used with the bar-code sticker shown in FIG. 2C, in accordance with an embodiment of the invention;

FIG. 4A schematically shows a sticker adapted to be mounted on a glucometer using Velcro, in accordance with an embodiment of the invention;

FIG. 4B schematically shows a glucometer adapted to be used with the sticker shown in FIG. 4A, in accordance with an embodiment of the invention

FIG. 4C schematically shows the sticker shown in FIG. 4A attached to the glucometer shown in FIG. 4B, in accordance with an embodiment of the invention;

FIG. 5 schematically shows a glucometer having mechanical clamps for mounting a sticker to thereto, in accordance with an embodiment of the invention;

FIG. 6 schematically shows a split-unit glucometer comprising a control unit and a separate sensor unit, in accordance with an embodiment of the invention;

FIG. 7 schematically shows the split-unit glucometer shown in FIG. 6 being used by a patient, in accordance with an embodiment of the invention;

FIG. 8 schematically shows a sticker for attaching a glucometer to a patient's skin that comprises a battery for providing power to a glucometer, in accordance with an embodiment of the invention; and

FIG. 9 schematically shows a sensor sticker comprising a light source and an acoustic transducer in accordance with an embodiment of the invention.

### DETAILED DESCRIPTION OF EXEMPLARY EMBODIMENTS

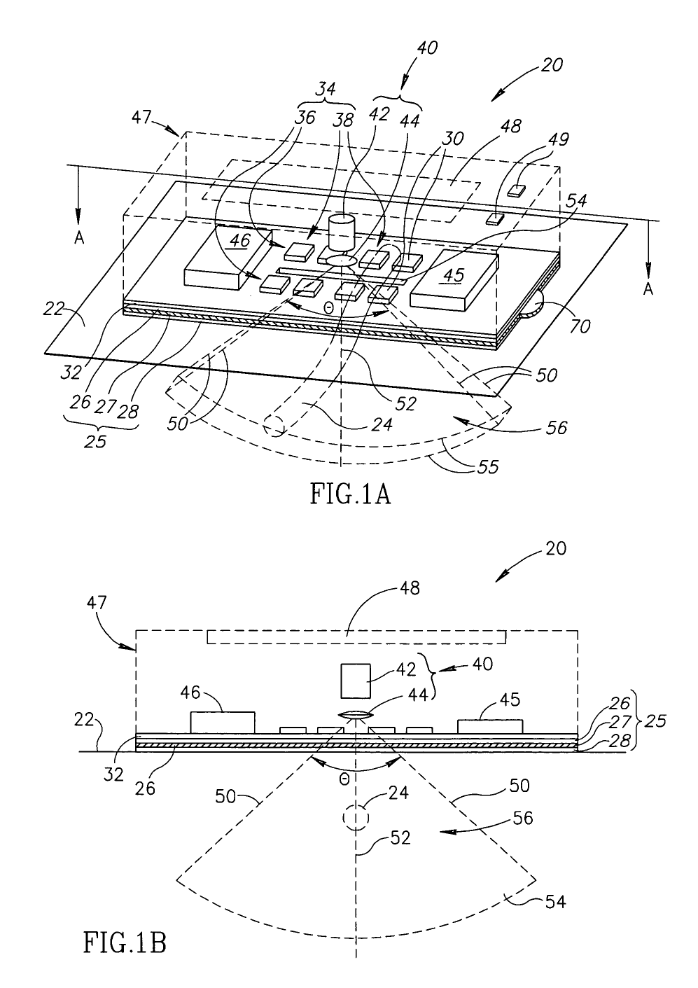

FIGS. 1A and 1B schematically show a perspective view and cross-section view respectively of a glucometer 20 in accordance with an embodiment of the present invention. The cross section view shown in FIG. 1B is taken in a plane indicated by a line “AA” in FIG. 1A. Glucometer 20 is shown attached to a region of skin 22 of a patient after it has been aligned with a blood vessel 24 located under the patient's skin in order to assay glucose in blood in the blood vessel.

Glucometer 20 comprises a plurality of acoustic transducers 30 mounted to a mounting panel 32, which are optionally configured in an array 34 of rows 36 and columns 38, and a light provider 40 comprising a light source 42 and optionally optics represented by a lens 44. By way of example, the number of transducers 30 in array 34 is four and the array has two rows 36 and two columns 38. A controller 46 controls light provider 40 and transducer array 34. The components of glucometer 20 are comprised in a housing 47 indicated by dashed lines.

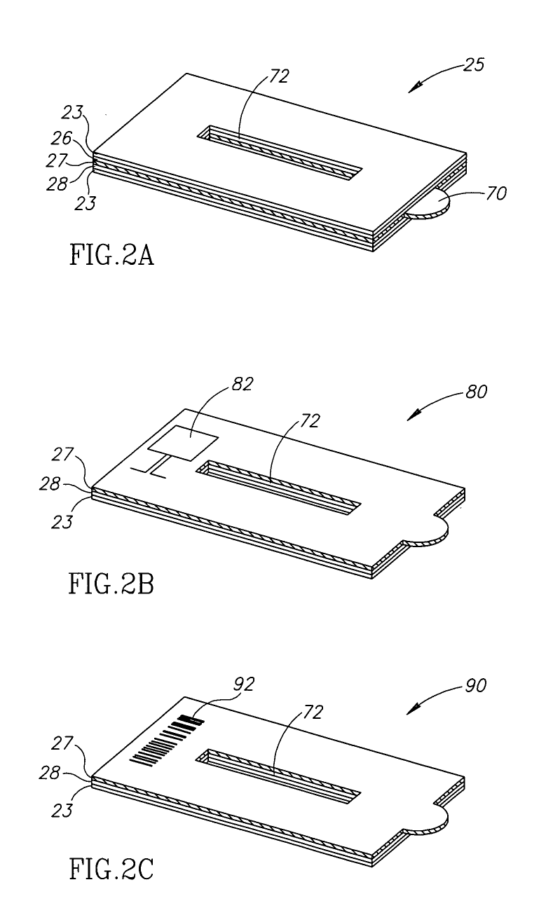

Optionally, a power source 45 for powering controller 46 and light source 42 is mounted inside housing 47. In some embodiments of the invention, power for controller 46 and light source 42 is provided by an external power source to which glucometer 20 is connected. Optionally, the external power source is mounted to the patient's body. Housing 47 optionally has a visual display screen 48 and control buttons 49 for interfacing with controller 46. Glucometer 20 is optionally attached to skin 22 by a suitable adhesive sticker 25 that bonds mounting panel 32 to skin 22. Sticker 25 and variations thereof, in accordance with embodiments of the invention are shown in FIGS. 2A-2C and discussed in detail below.

In some embodiments of the invention, mounting panel 32 is formed from a flexible piezoelectric material, such as PVDF and acoustic transducers 30 are integrally formed elements of the mounting panel. Each integrally formed acoustic transducer 30 comprises a region of mounting panel 32 sandwiched between a first electrode on a top surface of the mounting panel and a second electrode on a bottom surface of the mounting panel. Voltage generated between the first and second electrode of a transducer 30 responsive to acoustic energy incident on the transducer is used to sense the acoustic energy. Whereas first electrodes of transducers are substantially electrically isolated from each other, each second electrode may be a region of a same single large electrode optionally on the bottom surface of the mounting panel. For convenience of presentation, in FIGS. 1A and 1B and figures that follow, transducers 30 are shown as separate elements mounted on mounting panel 32.

Light from light source 42 is optionally shaped by lens 44 into a relatively thin fan shaped beam of light schematically indicated by dashed lines 50 and directed so that it is incident on mounting panel 32 between rows 36 of transducers 30. Fan beam 50 has a central axis 52 and a fan angle θ. To enable light in fan beam 50 to pass through mounting panel 32 and illuminate tissue below skin 22, mounting panel 32 is optionally formed from a material that is transparent to light in fan beam 50. Additionally or alternatively, mounting panel 32 is formed with a slot 54 through which light beam 50 passes.

Optionally, sticker 25 comprises a substrate 27 having on one side an adhesive layer 26 suitable for bonding the sticker to mounting panel 32 and an adhesive layer 28 suitable for bonding the sticker to skin 22. Optionally, sticker 25 is formed from a material that is transparent to light in fan beam 50. Additionally or alternatively, sticker 25 does not cover slot 54 so as not to interfere with passage of light through the slot.

Intensity of light in fan beam 50 and a number and configuration of transducers 30 in array 34 are such that photoacoustic waves stimulated by the light beam in tissue to a depth below skin 22 indicated by dashed “depth” lines 55 are generally detectable by the transducer array. A region 56 of the tissue in which photoacoustic waves that are detectable by transducer array 34 are stimulated is substantially bounded by the envelope of fan beam 50 and dashed depth lines 55. Region 56 is coincident with the field of view of glucometer 20 and will be referred to as “field of view 56”.

Since glucometer 20 is assumed to be aligned with blood vessel 24, the blood vessel passes substantially through axis 52 in a direction substantially perpendicular to the plane of fan beam 50. In accordance with an embodiment of the invention, lens 44 forms fan beam 50 having a fan angle θ large enough so that at an expected depth of blood vessel 24 below skin 22 a cross section of field of view 56 in the plane of the fan beam 50 is substantially larger than a typical cross section of the blood vessel.

Optionally, fan beam 50 is configured so that at a depth of blood vessel 24 below skin 22, fan beam 50 extends on either side of the blood vessel by a distance, hereinafter an “alignment margin”, equal to about 3 mm. For example, for a diameter of blood vessel 24 equal to about 1 mm and having an expected location about 2 mm below the surface of skin 22, fan beam 50 is optionally configured so that at about 2 mm below the skin, width of the fan beam in the plane of the fan beam is equal to or greater than about 7 mm and the fan beam has a fan angle θ equal to about 120°.

In some embodiments of the invention, a glucometer similar to glucometer 20 is configured to have an alignment margin different from about 3 mm. For example, for a glucometer similar to glucometer 20 that is to be used to monitor glucose levels in an athlete during exercise, displacements by which the glucometer might become misaligned may be expected to be greater than usual and the glucometer configured to have an alignment margin greater than about 3 mm. Optionally the alignment margin is equal to about 5 mm. For a bed-ridden patient a glucometer may have an alignment margin less than about 3 mm. Optionally, the alignment margin is equal to about 2 mm.

To align glucometer 20 with blood vessel 24 as shown in FIGS. 1A and 1B, glucometer 20 is placed on a region of skin 22 below which blood vessel 24 is expected to be located. A suitable gel or oil is optionally used to acoustically couple the glucometer to the skin. A control signal is input to glucometer 20 via interface buttons 49 instructing controller 46 to operate in an alignment mode and the glucometer is oriented so that the plane of fan beam 50 is substantially perpendicular to the length of the blood vessel. Optionally, controller 46 indicates orientation of the plane of fan beam 50 by generating a suitable icon on display screen 48.

The patient and/or a person aiding the patient, then moves glucometer 20 back and forth substantially in a direction perpendicular to the length of blood vessel 24. Optionally, during motion of glucometer 20, controller 46 controls transducer array 34 to image features below skin 22 and in particular blood vessel 24 with ultrasound using methods known in the art. In some embodiments of the invention, Doppler shifted ultrasound imaging methods known in the art are used to image blood vessel 24. Optionally, during motion of glucometer 20, controller 46 controls light provider 40 to illuminate tissue below skin 22 with light that stimulates photoacoustic waves in the tissue. Optionally, controller 46 controls light provider 40 to illuminate tissue below skin 22 with light at least one wavelength that is strongly absorbed by blood. Signals generated by transducer array 34 responsive to the photoacoustic waves are used to provide a “photoacoustic” image of features below skin 22 and in particular blood vessel 24.

Optionally, controller 46 generates a signal responsive to the ultrasound and/or photoacoustic image to aid a user of glucometer 20 to align the glucometer with the blood vessel. For example, controller 46 may control a LED and/or a small speaker (not shown) responsive to the image to provide an optical and/or audio signal indicating when glucometer 20 is aligned with blood vessel 24.

Optionally, controller 46 displays the ultrasound and/or photoacoustic image on screen 48 to facilitate aligning the glucometer with the blood vessel. For example, in some embodiments of the invention controller 46 displays the ultrasound or photoacoustic image on screen 48 together with a suitable fiducial mark representing the center of the field of view of glucometer 20. The patient, and/or the patient's aid, aligns glucometer 20 with blood vessel 24 responsive to a location in the image of blood vessel 24 relative to the fiducial mark.

Once the glucometer is substantially aligned with blood vessel 24, the position of the aligned glucometer on the patient's skin is optionally marked using any suitable marking device, such as a pen for marking skin with non-toxic ink. The patient then removes glucometer 20 from skin 22 and optionally applies sticker 25 to mounting panel 32 by pressing adhesive layer 26 of the sticker into contact with the mounting panel.

The patient and/or the patient's aid then repositions glucometer 20 on skin 22 responsive to the alignment marks with the adhesive layer 28 in contact with the skin and presses the glucometer to the skin to assure proper contact of the skin to the adhesive layer. Methods of aligning a glucometer with a blood vessel are described in PCT Publications WO 2004/107971 and WO 2005/067786 referenced above.

Once properly aligned, a control signal is input to the glucometer via interface buttons 49 instructing controller 46 to operate in an assay mode to assay glucose in blood vessel 24. In the assay mode controller 46 controls light provider 40 to illuminate region 56 with fan beam 50 at least one wavelength that is scattered and/or absorbed by glucose. Signals generated responsive to photoacoustic waves generated in blood in blood vessel 24 by the light are used to determine concentration of glucose in the blood. Any suitable method known in the art for processing the signals to determine the glucose concentration in the blood may be used. As noted above, exemplary methods for assaying glucose in blood in blood vessel 24 responsive to a photoacoustic effect are described in U.S. Pat. No. 6,846,288 referenced above and in PCT Publication WO 2004/086965.

As a result of the relatively large fan angle θ of fan beam 50 and its orientation substantially perpendicular to blood vessel 24, even if glucometer 20 becomes substantially misaligned with the blood vessel, the blood vessel will in general remain inside field of view 56 of the glucometer. As a result, degrees of misalignment typically encountered during operation of glucometer 20 will not in general substantially compromise satisfactory operation of the glucometer. (Displacements of glucometer 20 in a direction along the length of blood vessel 24 do not in general result in the blood vessel being displaced relative to the center of the field of view of the glucometer. On the other hand, displacements in a direction perpendicular to the length of blood vessel 24 do in general result in the blood vessel displacing relative to the center of field of view 56. However, because of the relatively large opening angle θ of fan beam 50, for typical misaligning displacements of glucometer 20 perpendicular to the length of blood vessel 24, in general the blood vessel remains within field of view 56 of the glucometer. It is expected that, for normal activity not including extreme physical exercise, glucometer 20 may become misaligned relative to blood vessel 24 during assay operation over a period of time equal to about a working day by distances of magnitude less than or equal to about 2 mm.)

In the course of use, a glucometer such as glucometer 20 will in general have to repeatedly be attached to and removed from a patient's skin and the use of stickers, such as sticker 25, in accordance with an embodiment of the invention, facilitates repeated attachment and removal of the glucometer. Optionally, every time glucometer 20 is attached to a patient's skin 22 a new sticker, such as sticker 25, in accordance with an embodiment of the invention, is attached to mounting panel 32 and used to attach the glucometer to the skin. Sticker 25 may have any of various configurations, in accordance with embodiments of the invention.

FIG. 2A schematically shows sticker 25 configured for bonding mounting panel 32 to skin 22, in accordance with an embodiment of the invention. As noted above, sticker 25 optionally comprises a substrate 27 having an adhesive layer 26 suitable for bonding with mounting panel 32 and an adhesive layer 28 that is suitable for bonding with skin 22. Adhesive layers 26 and 29 are covered with protective films 23 that protect the adhesive layers during storage prior to use. Protective films 23 are removed to use the sticker to bond mounting panel 32 to skin 22. Optionally, sticker 25 is formed with a “peeling” tab 70 that facilitates removal of glucometer 20 from skin 22 and the sticker from the glucometer. Optionally, neither side of peeling tab 70 is covered with an adhesive layer.

Optionally, substrate 27 and adhesive layers 26 and 28 are formed from materials that are substantially transparent to light in fan beam 50. As a result, when protective films 23 are removed and sticker 25 is functioning to bond mounting panel 32 to skin 22 the sticker enables light in fan beam 50 to pass substantially unobstructed to the skin. Additionally or alternatively sticker 25 is optionally formed with a slot that corresponds to slot 54 (FIG. 1A) so that when the sticker is adhered to mounting panel 32 light in fan beam 50 that passes through slot 54 in the mounting panel can pass unobstructed to skin 22. By way of example sticker 25 is shown formed with a slot 72.

In some embodiments of the invention, adhesive layer 28 does not cover all of substrate 27 but instead is absent from regions of the substrate which lie under transducers 30 when the sticker is adhered to mounting panel 32. The regions that are absent the adhesive are “pre-spread” with an acoustic gel, optionally in the form of a gel pad, which facilitates acoustic coupling of the transducers to skin 22 when glucometer 20 is adhered to the skin by the sticker. Prior to use of the sticker, the acoustic gel is protected by protective film 23 that covers adhesive layer 28. Optionally, adhesive layer 26 is similarly also absent from regions that lie beneath transducers 30 and the regions are pre-spread with an acoustic coupling gel. In some embodiments of the invention, sticker 25 is formed with at least one hole that is pre-filled with an acoustic coupling material and is located beneath transducers 30 when the sticker is adhered to panel 32. Prior to use of sticker 25 the acoustic gel filling is protected by protective films 23.

Adhesive layer 26 that adheres mounting panel 32 to skin 22 bonds to the skin with a force sufficient to reliably maintain glucometer 20 coupled to the skin and enables the glucometer to be “peeled” off the skin. Peeling glucometer 20 from skin 22 is conveniently performed, optionally, lifting and applying a pulling force to peeling tab 70. After removal of glucometer 20 from skin 22, substrate 27 is peeled off mounting panel 32, optionally using peeling tab 70, and the mounting panel cleaned of remnant adhesive using any suitable devices and methods known in the art. Prior to subsequent attachment of glucometer 20 to skin 22 a new sticker 25 is adhered to mounting panel 32.

In some embodiments of the invention, a sticker for adhering a glucometer or component thereof to a patient's skin is provided with at least one device for presenting an identifying ID code.

FIG. 2B schematically shows a sticker 80, in accordance with an embodiment of the invention, that is similar to sticker 25 but in addition comprises a radio frequency identification (RFID) transponder 82 in accordance with any suitable configuration known in the art coded with a suitable ID code. Transponder 82 is formed on substrate 27 of sticker 80 and for convenience of presentation adhesive and protective layer 26 and its protective layer 23 which overlay the transponder are not shown in the figure. Optionally, controller 46 determines when sticker 80 is replaced, and when it determines that a new sticker 80 is mounted to mounting panel 32, the controller transmits an interrogation signal that requests the sticker's RFID transponder to transmit a signal comprising an ID code. If the controller does not receive an ID signal comprising a proper code or receives an ID signal comprising an improper code, the controller determines that the sticker is unsuitable and/or unauthorized for use with glucometer 20 and prevents operation of the glucometer.

Various methods and devices may be used to configure controller 46 to determine when a sticker is replaced. For example, sticker 80 is optionally formed to close or to open an electronic or optical circuit or generate a predetermined change in a characteristic, e.g. an impedance change, of such a circuit when it is respectively mounted to or removed from mounting panel 32. To open or close an electrical or optical circuit, optionally the sticker comprises an electrically or optically conductive component that cooperates with and mates with electrical or optical leads comprised in mounting panel 32. Optionally, to change a characteristic of an electronic circuit the sticker comprises a capacitive or inductive element that respectively changes a resonant frequency of the circuit. To change a characteristic of an optical circuit, optionally sticker 80 comprises an element that changes reflectivity of a surface region of mounting panel 32. Controller 46 determines that a sticker is replaced when it senses that the circuit is opened and closed or the characteristic evidences a change. In some embodiments of the invention, controller 46 periodically interrogates sticker 80 to determine if the sticker responds with a valid ID and if not the controller interrupts operation of glucometer 20.

FIG. 2C schematically shows a sticker 90, in accordance with an embodiment of the invention, which is another variation of sticker 25 having a device for presenting an ID code. Sticker 90 is provided with a barcode 92 formed on substrate layer 27 that provides the sticker with an identifying ID (For convenience of showing barcode 92, layers 26 and 23 that overlay the substrate layer are not shown). Optionally, controller 46 of a glucometer, similar to glucometer 20 (FIGS. 1A, 1B) that is used with barcode sticker 90 comprises or is coupled to a barcode reader for reading sticker barcodes. Optionally, the glucometer comprises a light source such as a laser that controller 46 controls to scan sticker barcodes and mounting panel 32 is formed with a barcode window through which the controller scans the sticker bar codes. Optionally, adhesive layer 26 does not cover the bar code and/or is transparent to light provided by the light source. If the controller determines that the sticker bears an invalid bar code, it interrupts operation of the glucometer.

In some embodiments of the invention, a sticker is provided with an ID by at least one substance, such as for example a dye, toner or ink, which is incorporated in or on the sticker. The at least one substance has a known absorption and/or scattering cross section for light and/or a spatial configuration on or in the sticker, which encodes the ID. The absorption cross-section and/or spatial configuration is read to determine the ID by illuminating the at least one substance with light to stimulate photoacoustic waves in the at least one substance and thereby the sticker that are sensed by acoustic transducers, optionally, in the glucometer. For example, in an embodiment of the invention, a sticker similar to sticker 90 (FIG. 2C) is formed with a spatial pattern in which different substances having known absorption and/or scattering cross sections for light at different wavelengths are deposited in different locations of the pattern. The ID code is read by illuminating the different regions of the pattern with light at wavelengths appropriate for stimulating photoacoustic waves in the regions to determine if the “correct” substances are located in the different regions.

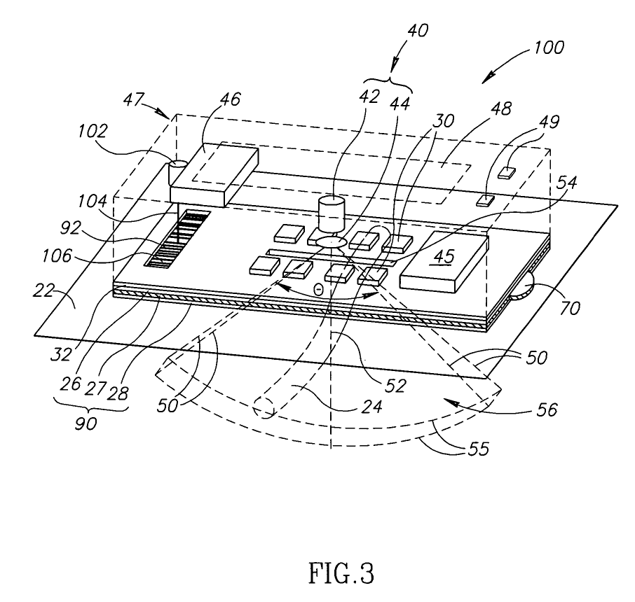

FIG. 3 schematically shows a glucometer 100 similar to glucometer 20 that is adapted to be used with bar-code sticker in accordance with an embodiment of the invention. Glucometer 100 optionally comprises the same components as glucometer 20 and in addition controller 46 optionally comprises a light source 102, such as a laser that provides a laser beam 104, for scanning sticker bar codes. Coupling panel 32 comprises a bar code window 106 through which controller 46 scans bar codes on stickers attached to the glucometer 100. In FIG. 3, by way of example, a sticker 90 is shown attached to glucometer 100 and the glucometer is schematically shown scanning bar code 92 of the sticker.

Whereas stickers 25, 80 and 90 are adhered to a glucometer by an adhesive layer 26 that bonds the stickers to mounting panel 32, in some embodiments of the invention a sticker is mounted to a glucometer using other methods.

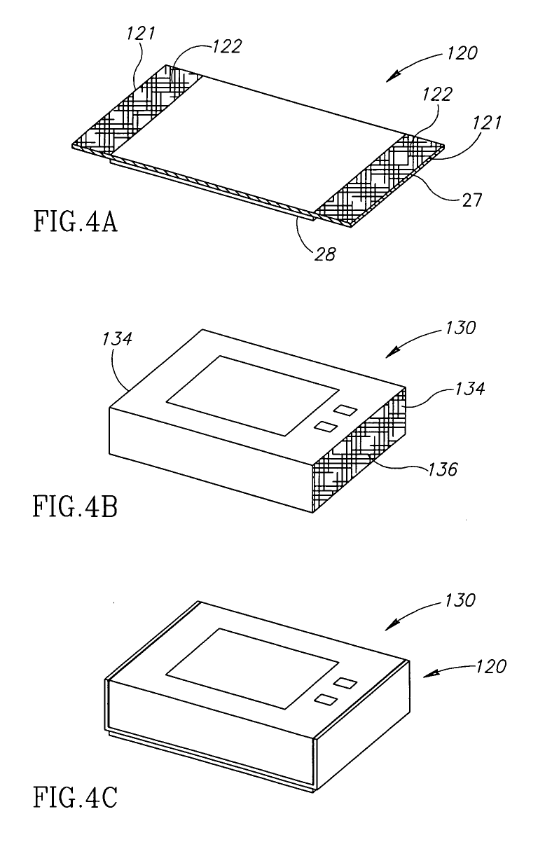

FIG. 4A schematically shows a sticker 120 that is mounted to a glucometer using Velcro fasteners, in accordance with an embodiment of the invention. Sticker 120 comprises a substrate 27 having on one side thereof an adhesive layer 28 for adhering the sticker to a patient's skin and along edges 121 of a second side thereof bands 122 of Velcro loops or hooks for attaching the sticker to the housing of a glucometer.

FIG. 4B schematically shows a glucometer 130 adapted to be used with sticker 120 in accordance with an embodiment of the invention. Only external features of glucometer 130 are shown in FIG. 4B. Glucometer 130 optionally has on its side surfaces 134, bands 136 of respectively Velcro hooks or loops that match and mate with bands 122 of Velcro loops or hooks on sticker 120.

Sticker 120 is mounted to glucometer 30 by optionally sticking a first one of Velcro bands 122 of the sticker to one of Velcro bands 136 of the glucometer. The sticker is then pulled taut in a direction towards the other Velcro band 136 of the glucometer and the second Velcro band 122 of the sticker is stuck to the second Velcro band of the glucometer. FIG. 4C schematically shows sticker 120 after it has been attached to glucometer 130. Except for the manner in which glucometer 130 is attached to a sticker, the glucometer is optionally similar to glucometer 20 (FIGS. 1A and 1B).

In some embodiments of the invention, a sticker is mounted to a glucometer similarly to the manner in which sticker 120 is mounted to glucometer 130 but instead of the sticker being held by Velcro fasteners to the glucometer the sticker is held to the glucometer mechanically.

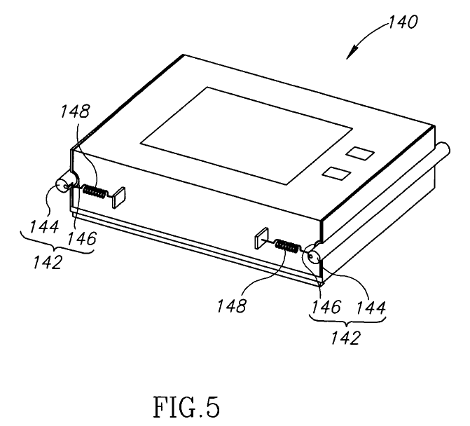

FIG. 5 schematically shows a glucometer 140, optionally similar to glucometers 20 and 130 but having mechanical clamps 142 for mounting a sticker to the glucometer. Each mechanical clamp 142 comprises an optionally cylindrical roller 144 and matching groove 146. Roller 144 is spring loaded using any devices and methods known in the art so that it seats in groove 146. By way of example, in FIG. 5 each roller 144 is resiliently urged to seat in its groove 146 by two elastic members schematically represented by springs 148 (only one of which is shown for each roller 144 in the perspective of the figure), one attached to each end of the roller. The other end of each elastic member is attached to the housing of glucometer 140. A sticker is mounted to glucometer 140 by displacing rollers 144 from their respective grooves 146 and inserting opposite edge regions of the sticker between the rollers and grooves and releasing the rollers so that they press the edge regions into the grooves to secure the sticker to the glucometer. FIG. 5 schematically shows a sticker 150 similar to sticker 25 secured to glucometer 140 by clamps 142.

Whereas glucometers 20, 100, 130 and 140 are “unitary glucometers” characterized in that all components of each glucometer are included in a single housing 47 (FIG. 1) in some embodiments of the invention, components of a glucometer are housed in a plurality of housings. Optionally, a “split-unit glucometer” comprises a control unit housed in its own control housing and at least one sensor unit housed in its own sensor housing. In some embodiments of the invention, the at least one sensor unit comprises a plurality of sensor units that are optionally mounted to different parts of a patient's body.

Optionally, each of the at least one sensor unit comprises, housed in its dedicated sensor housing, at least one light source that provides light for generating photoacoustic waves in tissue below a patient's skin and at least one acoustic transducer for detecting photoacoustic waves generated by the light provided by the light source. The control unit optionally comprises, housed in its dedicated housing, a controller for controlling the light source and acoustic transducers, transmitting and receiving signals to and from the transducers and light source, and optionally vetting sticker codes. The control housing also optionally comprises a power supply for the light source and transducers, a display screen and control buttons for interfacing with the controller. Splitting a glucometer into sensor and control units provides for greater flexibility in configuring the glucometer so that it can be mounted conveniently to a patient's body. Communication and transmission of power between the control and sensor units is optionally over wire and/or wireless electrical and/or optical channels.

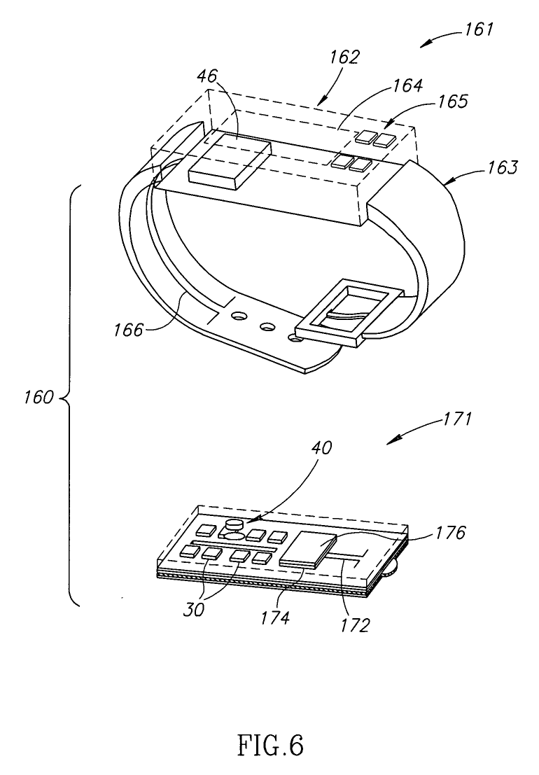

FIG. 6 schematically shows a “split-unit” glucometer 160 comprising a control unit 161 and a sensor unit 171, in accordance with an embodiment of the invention. Control unit 161 comprises a control housing 162 configured in a format of a wristwatch to be attached to a patient's wrist optionally with a “watch strap” 163. Control unit 161 comprises a controller 46, display screen 164 and a keyboard 165. Optionally, control unit 161 comprises at least one antenna 166 for communicating with and transmitting operating energy to sensor unit 171 over at least one wireless channel. Optionally, antenna 166 is comprised in watch strap 163.

Sensor unit 171 comprises a light provider 40 having at least one light source 42 and associated lens 44 and acoustic transducers 30, optionally configured similarly to the way in which light source 42 and transducers 30 are configured in glucometer 20 shown in FIG. 1A. Sensor unit 171 also, optionally comprises at least one antenna 172 for communicating with and receiving energy from control unit 161 over the at least one wireless channel, a storage unit 174 for storing energy that the sensor unit receives from control unit 161 and control circuitry 176. Sensor unit 171 is, optionally, mounted to a patient's body using a sticker, such as a sticker similar to sticker 25, in accordance with an embodiment of the invention.

Because glucometer control and operating functions are split between a control unit 161 and a sensor unit 171 in split-unit glucometer 160, the sensor unit can be made relatively small and does not have to be mounted on a patient's body so that it is readily accessible to the patient. As a result, sensor unit 171 can be conveniently mounted at different locations of the body that are optionally hidden from view by the patients clothes.

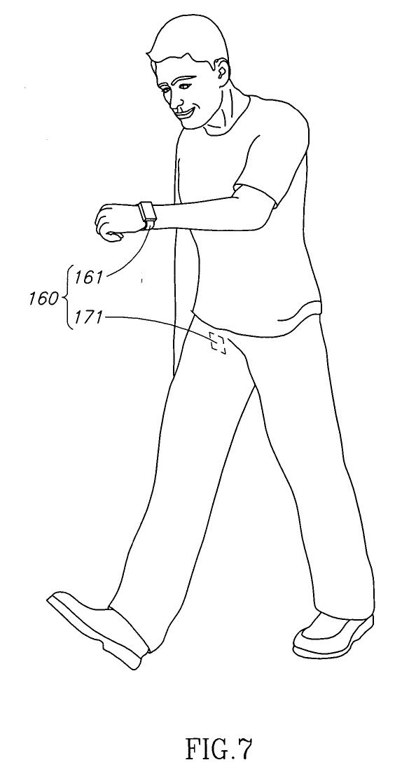

By way of example, FIG. 7 schematically shows split-unit glucometer 160 being used by a patient with sensor unit 171 mounted in the area of the patient's left groin over the femoral artery and control unit 161 strapped to the patient's wrist. In most standing and sitting positions, a person's wrist is generally in relative close proximity to the groin with the inside of the wrist facing towards the groin. Therefore, the locations of control unit 161 on the patient's wrist and sensor unit 171 on the patient's groin provide for relatively good wireless communication between the units. Provision of good wireless communication is also promoted by configuring control unit 161 in accordance with an embodiment of the invention so that when worn by a patient, control unit antenna 166 is located on watchstrap 163 (FIG. 6) so that it is located towards the inside of the patient's wrist. Sensor unit 171 may of course be mounted to locations of a patient's body other than in the region of the groin and can, by way of example, be mounted in the armpit, to the upper arm, the neck or thigh.

Whereas in split-unit glucometer 160, sensor unit 171 comprises both at least one light source and at least one transducer, a glucometer in accordance with an embodiment of the invention is not limited to comprising sensor units having both at least one light source and at least one transducer. In some embodiments of the invention, a glucometer sensor unit comprises either at least one light source or at least one acoustic transducer. A plurality of such different “optical” and “acoustic” sensor units may be mounted to a patient's body in different configurations to provide arrays of light sources and transducers that are advantageously adapted for sensing glucose concentrations at different locations of the body.

It is also noted that a split unit-glucometer may also be a self-aligning glucometer, in which the sensor unit comprises at least one motor and/or actuator controllable to move and realign the sensor unit if the glucometer determines that it has become misaligned. Any of the devices and configurations described in PCT Publication WO 2004/107971 for moving and realigning a glucometer referenced above may be used to configure the sensor unit so that it can realign itself.

In the above description of a sticker for attaching a glucometer or a unit of a split-unit glucometer to a patient's body in accordance with an embodiment of the invention, the sticker is described as optionally comprising, in addition to an adhesive layer for attaching the sticker to the skin, components that function to identify the sticker. A sticker in accordance with an embodiment of the invention may optionally comprise components in addition to, or in place of, a component or components noted above.

By way of example, FIG. 8 schematically shows a sticker 180 similar to sticker 80 (FIG. 2B) and comprising a battery 181, for providing power to a glucometer such as glucometer 20 or a part of a glucometer, such as sensor unit 171 of split-unit glucometer 160 (FIG. 6), in accordance with an embodiment of the invention. Battery 181 is formed on substrate 27 of sticker 180. For convenience of presentation, an adhesive layer optionally overlying substrate 27 and a protective film 23 that covers the adhesive layer is not shown in FIG. 8. Optionally battery 181 is produced, at least in part using a printing process or processes known in the art, such as described in U.S. Pat. No. 6,855,441 referenced above.

Battery 181 is conductively and/or inductively coupled to the glucometer or glucometer part to which it is attached using any of various methods and devices known in the art. By way of example, battery 181 is assumed to be conductively coupled to components of the glucometer or glucometer part and conducting contact leads 182 and 183 are formed on substrate 27 for providing electrical contact with corresponding contact leads in the glucometer or glucometer part when sticker 180 is attached thereto. Optionally, conducting contact leads 182 and 183 are formed using a printing technology known in the art such as a printing process described in U.S. Pat. No. 6,274,412, U.S. Pat. No. 6,300,932 and U.S. Pat. No. 6,521,489, the disclosures of which are incorporated herein by reference.

In some embodiments of the invention, a sticker, hereinafter a “sensor sticker”, having an adhesive layer for adhering the sticker to skin, comprises at least one light source for illuminating tissue and generating photoacoustic waves therein and acoustic transducers for sensing the acoustic waves. In some embodiments of the invention, a sensor sticker comprises a configuration of components so that the sticker, by itself, functions as a sensor unit of a split-unit glucometer. Such a sticker optionally comprises a substrate, such as for example a suitable polymer substrate on which a piezoelectric material, such as PVDF is deposited or printed and on which, a battery, at least one light source, electrodes and required circuit elements are formed. Printing of a piezoelectric material may be performed, for example, by screen-printing a slurry of powdered piezoelectric material as described in U.S. Pat. No. 5,639,508, the disclosure of which is incorporated herein by reference. Optionally, the battery, light source, electrodes and circuit elements are formed at least in part using printing technologies known in the art. Methods for printing optically active materials, such as light emitting elements, are discussed in U.S. Pat. No. 6,521,489 and U.S. Pat. No. 6,300,932. U.S. Pat. No. 6,545,291 discusses printing technologies and materials suitable for printing field effect transistors. All the U.S. patents referenced in the preceding sentence are incorporated herein by reference. Printing technologies for printing electrodes and conducting elements in a sensor sticker are optionally printed using a printing technology described in U.S. Pat. No. 6,274,412, U.S. Pat. No. 6,300,932 and U.S. Pat. No. 6,521,489 referenced above.

Optionally, a sensor sticker in accordance with an embodiment of the invention, is used with a control unit of a split-unit glucometer, such as control unit 161 of split unit glucometer 160 (FIG. 6). Optionally, a control unit and sensor sticker in accordance with an embodiment of the invention are configured so that the sensor sticker can be attached directly to the control unit to form a unitary glucometer similar to glucometer 20, 100, 130 or 140.

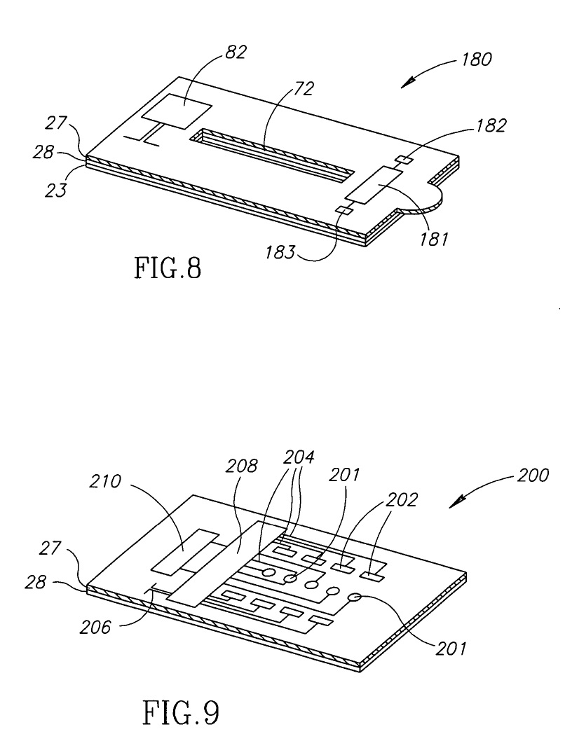

FIG. 9 schematically shows a sensor sticker 200, that by itself functions as a sensor unit in a split-sensor glucometer, such as split-unit glucometer 160 (FIG. 6), in accordance with an embodiment of the invention.

Sensor sticker 200 optionally comprises a substrate 27 having on one side an adhesive layer 28 suitable for sticking the sticker to the skin. Optionally, substrate 27 is printed with an array of light sources schematically indicated by circular icons 201 and an array of acoustic transducers indicated by square icons 202. Optionally, light sources 201 are printed on substrate 27 using a suitable printing method known in the art. Optionally, to enable light from light sources 201 to reach the skin of a patient to which sensor sticker 200 is attached, the substrate is transparent to light provided by light sources 201. Alternatively or additionally, substrate 27 is etched away beneath each light source 201. Alternatively or additionally, sticker 200 comprises an optical coupling agent in regions below light sources 201 to promote optical coupling of the light sources to the skin. Optionally, each acoustic transducer 202 comprises a piezoelectric material sandwiched between printed electrodes. The piezoelectric material is deposited on one of the electrodes between which it is sandwiched using any suitable deposition method known in the art. Optionally, sticker 200 comprises an acoustic coupling agent in regions below transducers 202 to promote acoustic coupling of the transducer to the skin. Lead and control lines 204 for signal transmission and powering and controlling light sources 201 and transducers 202 are, optionally, printed on substrate 27.

Optionally, sticker 200 comprises an antenna 206 for receiving signals from and transmitting signals to control unit 166 and control circuitry 208 for processing and using the signals. Optionally antenna 206 and at least portions of control circuitry 208 are formed using a suitable printing process. Optionally sticker 200 comprises a battery 210 formed on substrate 27, optionally using a printing process. In an embodiment of the invention, a protective insulating layer (not shown) is formed over substrate layer 27 to protect components of sticker 200 formed on the substrate.

It is noted, that whereas stickers in accordance with an embodiment of the invention have been shown and/or described being used with particular configurations of unitary and split-unit glucometers, a sticker in accordance with an embodiment of the invention may be used with glucometer configurations other than those shown. For example, a sticker in accordance with an embodiment of the invention may be used with a glucometer having a configuration or method of operation described in U.S. Pat. No. 6,846,288, PCT Publication WO 2004/107971 and/or PCT Publication WO 2005/067786 referenced above.

A sticker in accordance with an embodiment of the invention is not limited to use with a glucometer. A sticker similar to an exemplary embodiment of a sticker described herein may be used to attach medical devices and/or monitors other than a glucometer to a patient's body. For example, such a sticker may be used to attach a pulse or temperature monitor, in particular one that uses light to perform measurements, to the body or an optoacoustic sensor described in PCT application WO 2005/068973 the disclosure of which are incorporated herein by reference. The sticker may include light sources and light sensors that respectively provide and sense light at different wavelengths to monitor optical properties of the tissue. A sticker in accordance with an embodiment of the invention may also include, in addition to or in place of a component noted above, electrodes to measure an electrical property, such for example resistance, of a patient's tissue or body part.

It is further noted that a split-unit glucometer may have configurations and operating principles other than those of the exemplary embodiments described above. For example, a split-unit glucometer may employ any of the methods of operation described in PCT Publication WO 2004/107971 referenced above and/or have a configuration for its sensor unit based on a configuration of sensors and light sources described in any of the above referenced documents.

In addition, apparatus and methods in accordance with embodiments of the invention similar to those that are described above for assaying glucose may be used for assaying other analytes, for example urea, albumin, hemoglobin and uric acid, of the body.

In the description and claims of the present application, each of the verbs, “comprise” “include” and “have”, and conjugates thereof, are used to indicate that the object or objects of the verb are not necessarily a complete listing of members, components, elements or parts of the subject or subjects of the verb.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons of the art. The scope of the invention is limited only by the following claims.

1. Apparatus for assaying an analyte in a patient's body comprising:
at least one sensor unit mountable to a region of the patient's skin comprising at least one light source that illuminates a tissue region below the skin with light that generates photoacoustic waves therein and at least one acoustic transducer that generates signals responsive to the photoacoustic waves; and
a control unit configured to be mounted to a different part of the body and comprising a controller for controlling the at least one light source and at least one acoustic transducer and receiving the signals generated by the at least one acoustic transducer.

2. Apparatus according to claim 1 wherein each of the control unit and at least one sensor unit comprises a transmitter and receiver for communication over at least one channel.

3. Apparatus according to claim 2 wherein the at least one channel comprises a wireless channel.

4. Apparatus according to claim 3 wherein the at least one wireless channel comprises an electromagnetic wave channel.

5. Apparatus according to claim 4 wherein the electromagnetic wave channel comprises an optical channel.

5. Apparatus according to claim 3 wherein the at least one wireless channel comprises an acoustic channel.

6. Apparatus according to claim 2 wherein the at least one channel comprises a wire channel.

7. Apparatus according to claim 6 wherein the wire channel comprises an optical wave guide.

8. Apparatus according to claim 7 wherein the optical wave guide comprises an optical fiber.

9. Apparatus according to claim 2 wherein the control unit transmits operating energy to the at least one sensor over a channel of the at least one channel.

10. Apparatus according to claim 9 wherein the at least one sensor comprises a storage unit that stores energy that it receives from the control unit.

11. Apparatus according to claim 1 wherein the at least one sensor unit comprises a power supply.

12. Apparatus according to claim 1 wherein the at least one sensor unit comprises a removable sticker having an adhesive layer that adheres the at least one sensor unit to skin.

13. Apparatus according to claim 12 wherein the sticker comprises a battery.

14. Apparatus according to claim 12 wherein the battery is formed at least in part by printing.

15. Apparatus according to claim 13 wherein the sticker comprises the at least one light source.

16. Apparatus according to claim 12 wherein the sticker comprises the at least one light source.

17. Apparatus according to claim 16 wherein the at least one light source is formed on or in the sticker at least in part by printing.

18. Apparatus according to claim 15 wherein the sticker comprises the at least one acoustic transducer.

19. Apparatus according to claim 12 wherein the sticker comprises the at least one acoustic transducer.

20. Apparatus according to claim 19 wherein the at least one transducer is formed on or in the sticker at least in part by printing.

21. Apparatus according to claim 12 wherein the sticker comprises a region having an acoustic coupling gel that promotes acoustic coupling of the at least one transducer to skin.

22. Apparatus according to claim 18 wherein the sticker comprises a region having an acoustic coupling gel that promotes acoustic coupling of the at least one transducer to skin.

23. Apparatus according to claim 12 wherein the sticker comprises at least one device for presenting an identifying ID code.

24. Apparatus according to claim 23 wherein the at least one device comprises a bar code.

25. Apparatus according to claim 23 wherein the at least one device comprises circuitry that transmits the ID code when interrogated.

26. Apparatus according to claim 25 wherein the circuitry comprises a transponder.

27. Apparatus according to claim 26 wherein the transponder comprises a radio frequency ID transponder.

28. Apparatus according to claim 23 wherein the at least one device comprises at least one substance having a known absorption and/or scattering cross section that encodes the ID.

29. Apparatus according to claim 23 wherein the at least one device comprises at least one substance characterized by known absorption cross section that is formed on or in the sticker in a spatial configuration that encodes the ID.

30. Apparatus according to claim 23 wherein the controller comprises apparatus to determine whether the at least one device for presenting an identifying ID code presents an acceptable ID code and if it does not, the controller prevents operation of the glucometer.

31. Apparatus according to claim 1 wherein the control unit is mountable to the wrist.

32. Apparatus according to claim 1 wherein the sensor unit is configured to be mounted over the femoral artery.

33. Apparatus according to claim 1 wherein the analyte is glucose.

34. A method for assaying an analyte in a patient's body comprising:
mounting at least one sensor unit to a region of the patient's skin comprising at least one light source that illuminates a tissue region below the skin with light that generates photoacoustic waves therein and at least one acoustic transducer that generates signals responsive to the photoacoustic waves; and
mounting to a region of body different from where the sensor unit is mounted, a control unit comprising a controller for controlling the at least one light source and at least one acoustic transducer and processing the signals generated by the at least one acoustic transducer to assay the analyte.

35. A method according to claim 34 wherein mounting the at least one sensor unit comprises mounting the unit over a femoral artery.

36. A method according to claim 34 wherein mounting the control unit comprises mounting the unit on the wrist.
